(19) United States
(12) Patent Application Publication
(10) Pub. No.: US 2025/0136863 A1
(43) Pub. Date: May 1, 2025

(54) GRAPHENE OXIDE (GO)-SILVER NANOPARTICLES (AgNPs)-Eu3+ FLUORESCENT PROBE, PAPER-BASED SENSOR, AND PREPARATION METHOD AND USE THEREOF

(71) Applicant: Henan Agricultural University, Zhengzhou City (CN)

(72) Inventors: Yaodi ZHU, Zhengzhou City (CN); Miaoyun LI, Zhengzhou City (CN); Lijun ZHAO, Zhengzhou City (CN); Jiaqi TIAN, Zhengzhou City (CN); Qiancheng TU, Zhengzhou City (CN); Lingxia SUN, Zhengzhou City (CN); Yanxia LIU, Zhengzhou City (CN); Gaiming ZHAO, Zhengzhou City (CN); Chuang LI, Zhengzhou City (CN); Wenyan DING, Zhengzhou City (CN)

(21) Appl. No.: 18/926,617

(22) Filed: Oct. 25, 2024

(30) Foreign Application Priority Data
Oct. 30, 2023 (CN) 202311421447.1

(51) Int. Cl. C09K 11/65; G01N 21/64; G01N 33/04

(52) U.S. Cl. CPC C09K 11/65; G01N 21/6447; G01N 33/04

(57) ABSTRACT

A graphene oxide (GO)-silver nanoparticles (AgNPs)-Eu³⁺ fluorescent probe, a paper-based sensor, and a preparation method and use thereof are provided. The paper-based sensor is developed based on a GO-AgNPs-Eu³⁺ fluorescent probe-loaded polyvinylidene fluoride (PVDF) microporous membrane, which is further combined with a smartphone. The GO-AgNPs-Eu³⁺ fluorescent probe shows high selectivity for 2,6-dipicolinic acid (DPA), strong anti-interference ability, and low limit of detection (LOD) for the DPA and spores. The paper-based sensor realizes quantitative detection of the DPA and the spores through red, green, and blue (RGB) changes of the smartphone, and detection results are verified by an actual amount of the spores in milk and water. The rapid detection of foodborne spores is achieved through a dual-function platform based on fluorescence of the GO-AgNPs-Eu³⁺ fluorescent probe and the paper-based sensor.

### CROSS REFERENCE TO RELATED APPLICATION

This patent application claims the benefit and priority of Chinese Patent Application No. 202311421447.1, filed with the China National Intellectual Property Administration on Oct. 30, 2023, the disclosure of which is incorporated by reference herein in its entirety as part of the present application.

### TECHNICAL FIELD

The present disclosure relates to preparation of a graphene oxide (GO)-silver nanoparticles (AgNPs)-Eu³⁺ fluorescent probe, and development of a paper-based sensor prepared using a GO-AgNPs-Eu³⁺ fluorescent probe-loaded polyvinylidene fluoride (PVDF) microporous membrane combined with a smartphone. The present disclosure specifically relates to a preparation method of a fluorescent probe and a development method of a paper-based sensor for detection of a foodborne spore, belonging to the technical field of nanomaterials and food safety detection.

### BACKGROUND

Under environmental stress, spore-forming bacteria undergo a series of temporal and spatial gene expressions, and finally form dormant bodies in round or oval shape with strong stress resistance, namely spores, to resist adverse environments. The spores can survive for several years in a dormant state and sense environmental changes at all times. Once it is suitable for growth, the spores can germinate to form vegetative bodies with normal physiological activity and then produce toxins, causing food to rot and deteriorate or even foodborne diseases, bringing great economic losses to the food industry. Moreover, the accompanying strong pathogenicity and even lethality pose a great threat to human health and the development of enterprises. Therefore, it is a key to controlling foodborne spores by developing rapid, sensitive, highly reproducible, timely, and effective detection technologies.

Spores are mainly composed of DNA, different receptor proteins, peptidoglycan, and 2,6-dipicolinic acid (DPA). The DPA, one of the main components of spores (accounting for 15% of a dry weight of the spores), is present in the sporoplasm and serves as a unique substance that can be used as a biomarker for identifying the spores. In order to timely assess the risk of spore contamination, different technologies have been developed for highly sensitive detection of spores at present, such as polymerase chain reaction (PCR), immunoassay, mass spectrometry (MS), electrochemistry, and surface-enhanced Raman spectroscopy (SERS), but they all have some inevitable defects. Among the reported technologies for fluorescent detection of spore DPA, lanthanide ions have a strong specific chelation affinity with the spore biomarker DPA, and display fluorescence at a unique wavelength through absorption energy transfer emission (AETE), achieving rapid detection of DPA. Moreover, lanthanum-based fluorescent probes have a long fluorescence lifetime and a narrow linear emission band, which help to effectively eliminate interference from background signals to improve the accuracy of fluorescence detection.

Therefore, a GO-AgNPs-Eu³⁺ nanomaterial is innovatively constructed as a fluorescent probe to detect the spore biomarker DPA based on the characteristics of lanthanide elements and graphene oxide (GO)-silver nanoparticles (AgNPs). In addition, the fluorescent probe is loaded with a polyvinylidene fluoride (PVDF) microporous membrane and then combined with a smartphone to develop a paper-based sensor based on the GO-AgNPs-Eu³⁺ nanomaterial. The paper-based sensor can be used for rapid visual detection of the spore DPA, and spores in milk and water are verified based on a dual-functional platform of fluorescence and the paper-based sensor, thereby achieving on-site rapid quantification and real-time online analysis of the spores.

### SUMMARY

In view of the problems existing in the prior art, the present disclosure provides a GO-AgNPs-Eu³⁺ fluorescent probe, a paper-based sensor and a preparation method thereof, and use in foodborne spore detection. In the present disclosure, the GO-AgNPs-Eu³⁺ fluorescent probe shows high selectivity for DPA, strong anti-interference ability, and low detection limit (LOD) for DPA and spores. A fluorescence color of the paper-based sensor changes from blue to red as a concentration of the DPA increases, and quantitative detection of the DPA and spores is achieved through red, green, and blue (RGB) changes of a smartphone, and detection results are verified by an actual amount of the spores in milk and water. The rapid detection of foodborne spores is achieved through a dual-function platform based on fluorescence of the GO-AgNPs-Eu³⁺ fluorescent probe and the paper-based sensor.

In order to solve the above technical problems, the present disclosure uses DPA as a biomarker of spores to explore an interaction mechanism between the GO-AgNPs-Eu³⁺ fluorescent probe and the DPA. Aiming at the selectivity and anti-interference ability of DPA, a smartphone-assisted, GO-AgNPs-Eu³⁺ nanomaterial-loaded, and portable visual paper-based sensor is designed for on-site rapid quantification and real-time online analysis of the spores.

Specifically, the present disclosure adopts the following technical solutions:

The present disclosure provides a preparation method of a GO-AgNPs-Eu³⁺ fluorescent probe, including the following steps:
(1) preparing GO;
(2) preparing ethylenediaminetetraacetic acid dianhydride (EDTAD)-modified GO-AgNPs using the GO; and
(3) preparing the GO-AgNPs-Eu³⁺ fluorescent probe: dispersing the EDTAD-modified GO-AgNPs in ultrapure water, adding an Eu(NO₃)₃·6H₂O aqueous solution dropwise in an ultrasonic environment and stirring for a period of time; collecting a product obtained by centrifugation, washing the product with deionized water for multiple times, and then resuspending the product in the ultrapure water to obtain the GO-AgNPs-Eu³⁺ fluorescent probe.

Further, a preparation process of the GO in step (1) includes: adding 0.5 g of a graphite flake and 0.25 g of sodium nitrate into 50 mL of concentrated sulfuric acid, cooling an obtained mixture to 0° C., adding potassium permanganate and stirring at a room temperature for 30 min, adding 25 mL of the ultrapure water and 2 mL of hydrogen peroxide with a mass fraction of 30% at 98° C. to allow heat preservation for 15 min, washing an obtained reaction product with 0.1 M hydrochloric acid and water in sequence, and then vacuum drying at 60° C. for 12 h to obtain the GO.

Further, a preparation process of the EDTAD-modified GO-AgNPs in step (2) includes: adding 200 μL of a 0.5 mg/mL GO aqueous solution into 19 mL of the ultrapure water and stirring continuously (500 r/min) for 15 min (pH=9), and then adding 0.3 mL of a 10 mM silver nitrate solution and stirring continuously for 30 min; slowly adding 50 μL of a 0.01 M sodium borohydride solution and stirring continuously for 4 h; heating an obtained solution to 70° C., adding 250 mL of the ultrapure water, 25 mL of a 1,2-bis(2-aminoethoxy)ethane solution, and 500 mg of potassium hydroxide in sequence and stirring vigorously (2,000 r/min) for 24 h; adding 50 mL of a 0.5 M sodium borohydride solution to allow a reaction at 70° C. for 2 h, and then collecting a precipitate obtained by centrifugation and washing the precipitate thoroughly with water; dispersing 10 mg of the precipitate ultrasonically in 5 mL of a sodium bicarbonate buffer (pH=9.6, 0.1 M), adding 80 mg of EDTAD and stirring for 2 h, and subjecting obtained nanoparticles (NPs) to centrifugal separation, washing with the sodium bicarbonate buffer 4 times and then deionized water 2 times in sequence, and vacuum drying at 60° C. for 12 h to obtain the EDTAD-modified GO-AgNPs.

Further, the Eu(NO₃)₃·6H₂O aqueous solution has a concentration of 0.01 M, 5 mL of the ultrapure water and 5 mL of the Eu(NO₃)₃·6H₂O aqueous solution are required based on 10 mg of the EDTAD-modified GO-AgNPs, the stirring is conducted for 3 h, and a solution of the GO-AgNPs-Eu³⁺ fluorescent probe has a concentration of 2 mg/mL in step (3).

The present disclosure further provides a GO-AgNPs-Eu³⁺ fluorescent probe prepared by the preparation method.

The present disclosure further provides a preparation method of a paper-based sensor, including: immersing a PVDF microporous membrane as a substrate into a Tris buffer of the GO-AgNPs-Eu³⁺ fluorescent probe, conducting incubation for a period of time, and then drying naturally in the air to obtain a PVDF microporous membrane-modified paper-based sensor.

Further, the Tris buffer has a concentration of 10 mM and a pH value of 7.0, and the GO-AgNPs-Eu³⁺ fluorescent probe has a concentration of 5 mg/mL and is incubated for 20 min in the Tris buffer.

The present disclosure further provides use of the GO-AgNPs-Eu³⁺ fluorescent probe in rapid detection of a foodborne spore, and use of a PVDF microporous membrane-modified paper-based sensor prepared by the preparation method in rapid detection of a foodborne spore, where the PVDF microporous membrane-modified paper-based sensor loaded with the GO-AgNPs-Eu³⁺ fluorescent probe is combined with a smartphone to allow on-site visual detection of spore DPA, and spores in milk and water are verified through a dual-function platform based on fluorescence and the paper-based sensor.

The present disclosure provides use of the GO-AgNPs-Eu³⁺ fluorescent probe and the PVDF microporous membrane-modified paper-based sensor in detection of a foodborne spore.

The use based on the fluorescent probe includes the following three aspects: (1) studies on the selectivity, anti-interference ability, and interaction between the GO-AgNPs-Eu³⁺ fluorescent probe and the DPA; (2) detection of the DPA as a biomarker of four representative spores (C. sporogenes spores, B. subtilis spores, B. cereus spores, and B. thuringiensis spores); (3) development of a PVDF microporous membrane-modified sensor based on the GO-AgNPs-Eu³⁺ nanomaterial, which is combined with a smartphone to allow on-site visual detection of spore DPA, and spores in milk and water are verified through a dual-function platform based on fluorescence and the paper-based sensor.

Compared with the prior art, the present disclosure has the following beneficial effects:

The present disclosure discloses a preparation method of a GO-AgNPs-Eu³⁺ fluorescent probe, and provides a development method of a paper-based sensor with a GO-AgNPs-Eu³⁺ fluorescent probe-loaded PVDF microporous membrane combined with a smartphone. The GO-AgNPs-Eu³⁺ fluorescent probe shows high selectivity for DPA, strong anti-interference ability, and low LOD for the DPA and spores. The paper-based sensor realizes quantitative detection of the DPA and the spores through RGB changes of the smartphone, and detection results are verified by an actual amount of the spores in milk and water. The rapid detection of foodborne spores is achieved through a dual-function platform based on fluorescence of the GO-AgNPs-Eu³⁺ fluorescent probe and the paper-based sensor.

### BRIEF DESCRIPTION OF THE DRAWINGS

FIGS. 1A-1L show the characterization of a GO-AgNPs-Eu³⁺ nanomaterial; where FIG. 1A is a UV-visible spectrum; FIG. 1B is X-ray diffraction (XRD) characterization of the GO-AgNPs-Eu³⁺ nanomaterial; FIG. 1C is full-scan X-ray photoelectron spectroscopy (XPS) of the GO-AgNPs-Eu³⁺ nanomaterial; FIG. 1D is the XPS spectrum of C1s; FIG. 1E is the XPS spectrum of Ag3d; FIG. 1F is the XPS spectrum of Eu3d; FIG. 1G and FIG. 1H are scanning electron microscopy (SEM) images of the GO-AgNPs-Eu³⁺ nanomaterial; FIG. 1I, FIG. 1J, FIG. 1K, and FIG. 1L are elemental mapping diagrams of energy dispersive spectrometry (EDS) of the GO-AgNPs-Eu³⁺ nanomaterial;

FIG. 2A is a fluorescence spectrum of the GO-AgNPs-Eu³⁺ nanomaterial; FIG. 2B is fluorescence intensity comparison of the GO-AgNPs-Eu³⁺ nanomaterial at 616 nm; FIG. 2C is a fluorescence spectrum after adding DPA of different concentrations (0, 0.25, 0.5, 1.0, 2.0, 5.0, 7.5, 15.0, 20.0, 25.0, 30.0, 35.0, 40.0, and 45.0 μM) into the GO-AgNPs-Eu³⁺ nanomaterial; FIG. 2D is a standard curve of fluorescence intensity and DPA concentration based on the GO-AgNPs-Eu³⁺ nanomaterial; FIG. 2E is selectivity and anti-interference experiment of the GO-AgNPs-Eu³⁺ nanomaterial for DPA (where gray bar graph shows adding 200 μL of anti-interference solution into the GO-AgNPs-Eu³⁺ nanomaterial, and black bar graph shows the subsequent addition of 30 μL of DPA); FIG. 2F shows the RGB changes and differences of the DPA and various interfering compounds under 254 nm UV irradiation;

FIG. 3A, FIG. 3C, FIG. 3E, and FIG. 3G are fluorescence spectra of different concentrations of C. sporogenes spores, B. subtilis spores, B. cereus spores, and B. thuringiensis spores, respectively; FIG. 3B, FIG. 3D, FIG. 3F, and FIG. 3H are fluorescence standard curves of different concentrations of C. sporogenes spores, B. subtilis spores, B. cereus spores, and B. thuringiensis spores, respectively; and

FIG. 4A shows a complete release process of DPA from B. subtilis spores treated at 121° C. for 30 min; FIG. 4B is a simplified flowchart of DPA detection based on GO-AgNPs-Eu³⁺ nanomaterial test strips and smartphone sensors; FIG. 4C is a RGB-changed image and a linear relationship diagram (by plotting a linear relationship between R/B intensity and concentration) of DPA solutions (0 μM to 400 μM) and spore solutions (10¹ cfu/mL to 10⁷ cfu/mL) of a designed visual paper-based sensor under 254 nm handheld UV lamp.

### DETAILED DESCRIPTION OF THE EMBODIMENTS

The present disclosure will be further described below with reference to the specific examples and drawings. It should be understood that these examples are only intended to describe the present disclosure, rather than to limit the scope of the present disclosure. Those skilled in the art may make some non-essential improvements and adjustments to the present disclosure according to the content of the present disclosure.

### Example 1

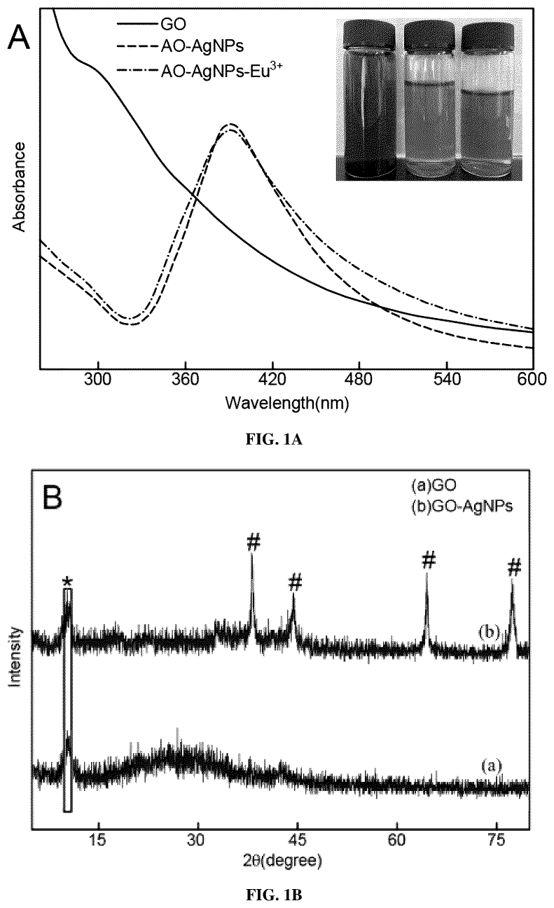

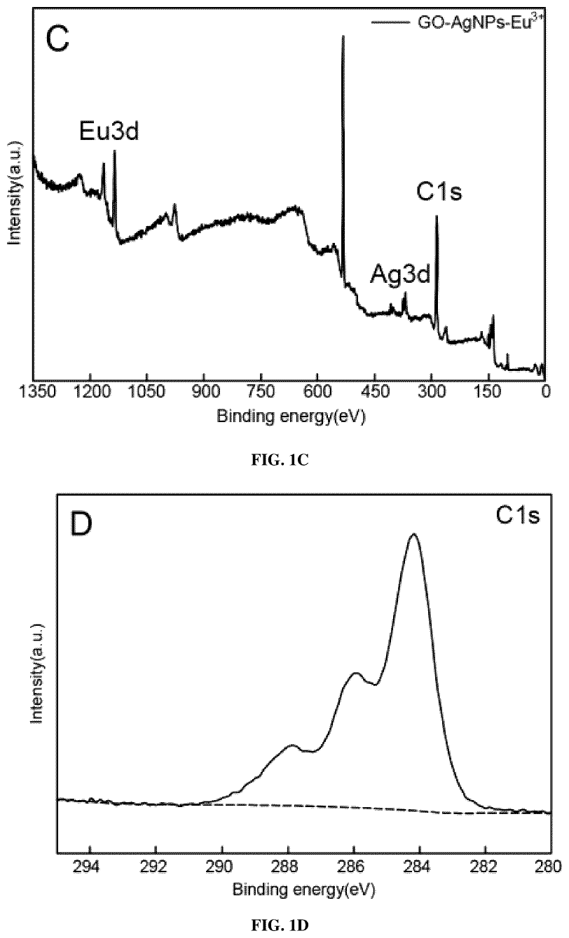

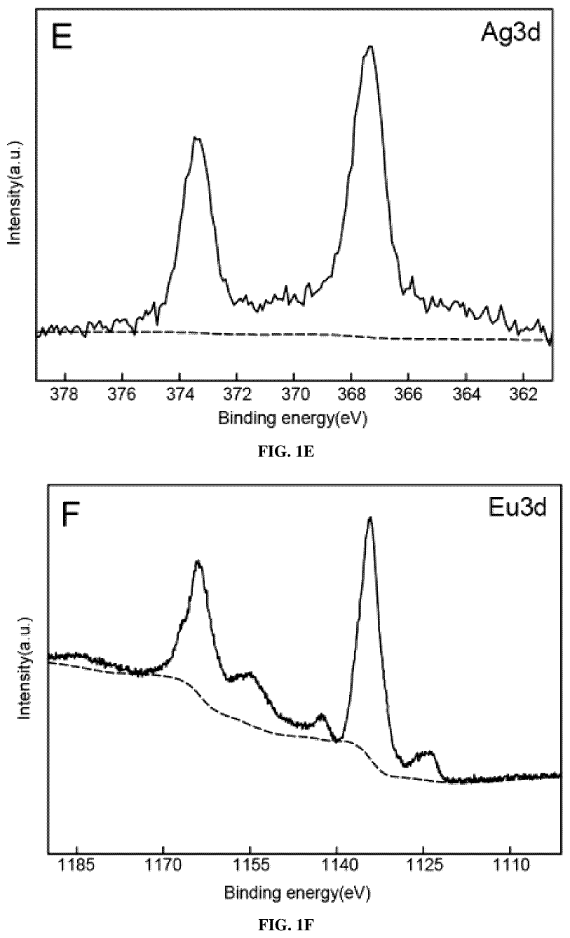

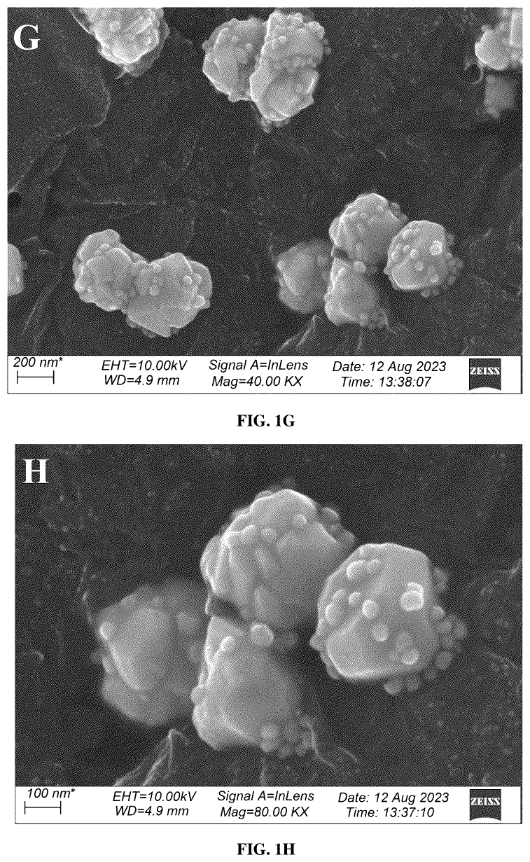

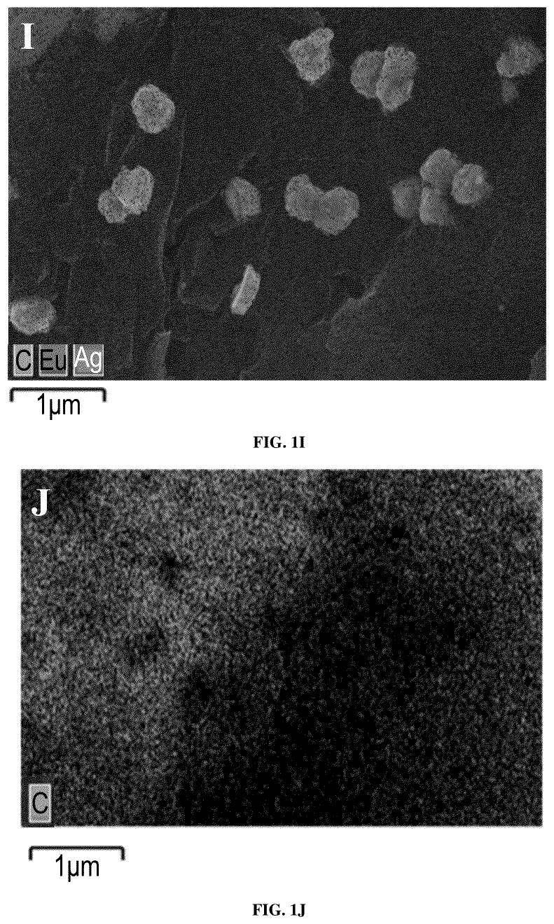

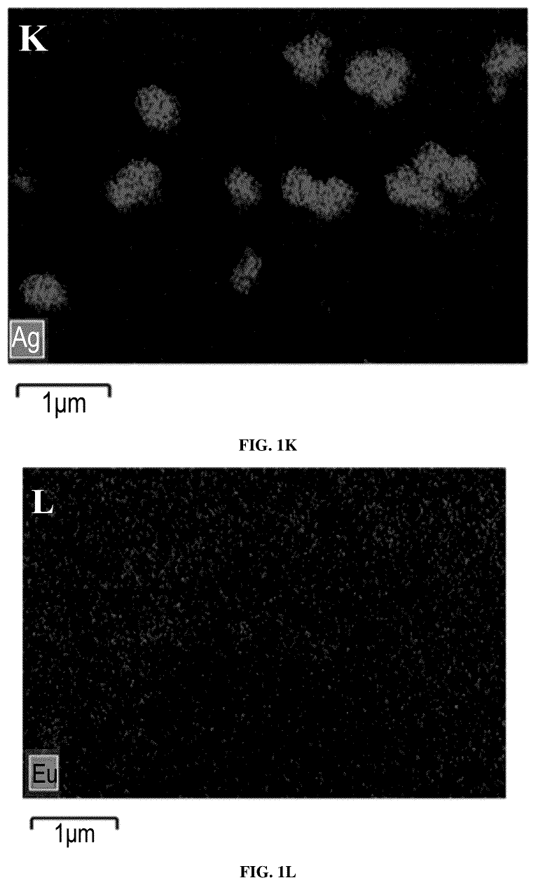

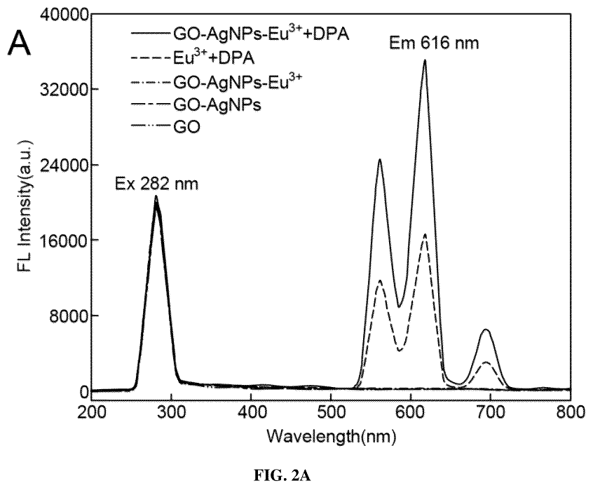

As shown in FIGS. 1A-1L, preparation and characterization of the GO-AgNPs-Eu³⁺ fluorescent probe included the following steps:

(1) Preparation of GO: 0.5 g of a graphite flake and 0.25 g of sodium nitrate were added into concentrated sulfuric acid, an obtained mixture was cooled to 0° C., potassium permanganate was added and stirred at a room temperature for 30 min, ultrapure water and 30% hydrogen peroxide were added at 98° C. to allow heat preservation for 15 min, an obtained reaction product was washed with 0.1 M hydrochloric acid and water in sequence, and then vacuum drying was conducted at 60° C. for 12 h to obtain the GO.

(2) Preparation of EDTAD-modified GO-AgNPs: 200 μL of a 0.5 mg/mL GO aqueous solution was added into 19 mL of the ultrapure water and stirred continuously for 15 min (pH=9), and then 0.3 mL of a 10 mM silver nitrate solution was added and stirred continuously for 30 min; 50 μL of a 0.01 M sodium borohydride solution was slowly added and stirred continuously for 4 h; an obtained solution was heated to 70° C., 250 mL of the ultrapure water, 25 mL of a 1,2-bis(2-aminoethoxy)ethane solution, and 500 mg of potassium hydroxide were added in sequence and stirred vigorously for 24 h; 50 mL of a 0.5 M sodium borohydride solution was added to allow a reaction at 70° C. for 2 h, and then a precipitate obtained by centrifugation was collected and the precipitate was washed thoroughly with water; 10 mg of the precipitate was dispersed ultrasonically in 5 mL of a sodium bicarbonate buffer (pH=9.6, 0.1 M), 80 mg of EDTAD was added and stirred for 2 h, and obtained nanoparticles (NPs) were subjected to centrifugal separation, washing with the sodium bicarbonate buffer 4 times and then deionized water 2 times in sequence, and vacuum drying at 60° C. for 12 h to obtain the EDTAD-modified GO-AgNPs.

(3) Preparation of the GO-AgNPs-Eu³⁺ fluorescent probe: 10 mg of the GO-AgNPs were dispersed in 5 mL of water, 5 mL of Eu(NO₃)₃·6H₂O (0.01 M) was added dropwise under ultrasonication and then stirred for 3 h. A product obtained by centrifugation was collected, washed several times with deionized water, and resuspended in 5 mL of ultrapure water to obtain 2 mg/mL of a GO-AgNPs-Eu³⁺ fluorescent probe solution.

The GO-AgNPs-Eu³⁺ fluorescent probe prepared in the above steps was characterized. As shown in FIGS. 1A-1L, where FIG. 1A was a UV-visible spectrum; FIG. 1B was XRD characterization of the GO-AgNPs-Eu³⁺ nanomaterial; FIG. 1C was full-scan XPS of the GO-AgNPs-Eu³⁺ nanomaterial; FIG. 1D was the XPS spectrum of C1s; FIG. 1E was the XPS spectrum of Ag3d; FIG. 1F was the XPS spectrum of Eu3d; FIG. 1G and FIG. 1H were SEM images of the GO-AgNPs-Eu³⁺ nanomaterial; FIG. 1I, FIG. 1J, FIG. 1K, and FIG. 1L were elemental mapping diagrams of EDS of the GO-AgNPs-Eu³⁺ nanomaterial.

The results showed that GO had a broad plasma resonance around 300 nm. When sodium borohydride was added to reduce and synthesize GO-AgNPs, the oxygen functional groups of GO were masked, resulting in the disappearance of the absorption peak. However, a characteristic absorption peak caused by AgNPs was observed at 390 nm, and the color of the mixture turned into the color of AgNPs solution. After the EDTA ligand on GO-AgNPs bound to Eu³⁺, the diameter of NPs increased, resulting in the broadening of the ion resonance absorption peak, which could preliminarily determine the formation of GO-AgNPs-Eu³⁺ nanomaterial. AgNPs were well dispersed on the graphene surface, granular Ag elements were distributed on the GO surface, and Eu was evenly dispersed on the surface of GO-AgNPs.

In addition, the crystal form of GO-AgNPs-Eu³⁺ nanomaterial was characterized by XRD. As shown in FIG. 1B, curve (a) showed that a sharp diffraction peak (2θ) of GO was distributed at 10.7°; curve (b) showed that the diffraction peaks (2θ) of GO-AgNPs were distributed at 10.7°, 38.24°, 44.4°, 64.5°, and 77.46°, corresponding to the (001) of GO, and the (111), (200), (220), and (311) crystal planes of a silver cubic structure, respectively, and the results were consistent with those in a powder diffraction file database card (JCPDS 75-1609). Moreover, the curve (b) had both the diffraction peak (2θ) of GO and the diffraction peak (2θ) of AgNPs. The deconvoluted XPS C1s region showed three significant peaks at 284.1, 285.8, and 287.8 eV, corresponding to sp² carbon, O—C—O, and O—C═O bonds, respectively. The deconvolution of the XPS spectrum of Ag3d showed that the binding energies of Ag3d5/2 and Ag3d3/2 had two peaks at 267.8 and 373.8 eV, respectively. The formed AgNPs-Eu³⁺ nanomaterial contained a metallic state (Ag0), and two peaks could be observed at 1,134.1 eV and 1,163.7 eV, corresponding to Eu (II) 3d5/2 and Eu (III) 3d5/2, respectively. These characterizations could fully prove the successful synthesis of GO-AgNPs-Eu³⁺ nanomaterial.

### Example 2

Use of the GO-AgNPs-Eu³⁺ fluorescent probe prepared in Example 1 in the detection of DPA

200 μL of a DPA sample and 2 μL of the GO-AgNPs-Eu³⁺ nanomaterial (with concentration of 2 mg/mL) were added into 500 μL of Tris buffer (10 mM, pH=7.0). The emission spectrum was collected by a Hitachi F4600 fluorescence spectrophotometer, where all tests were done at room temperature with a scanning rate of 1,200 nm·min⁻¹. The fluorescence intensity was measured at an excitation wavelength of 282 nm and an emission wavelength of 616 nm, where slits for excitation and emission were fixed at 10 nm, the fluorescence spectrum was scanned at 700 V voltage, and the number of experimental repetitions was n=3.

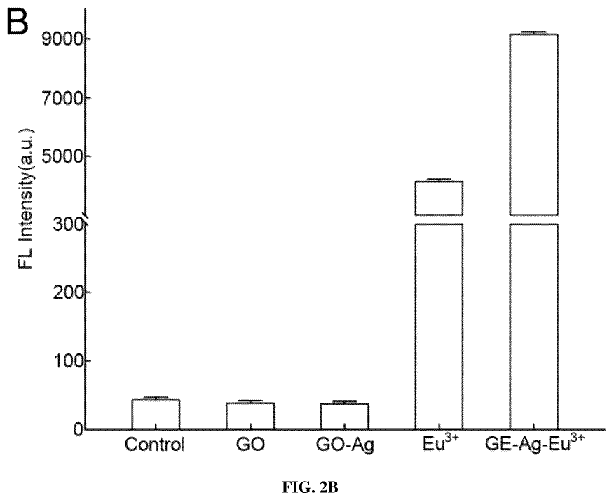

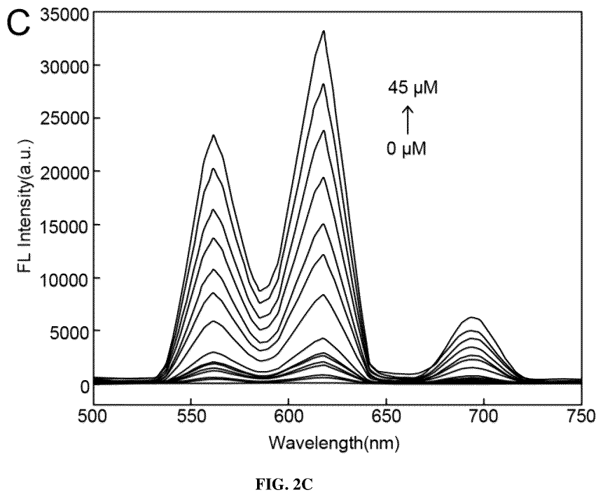

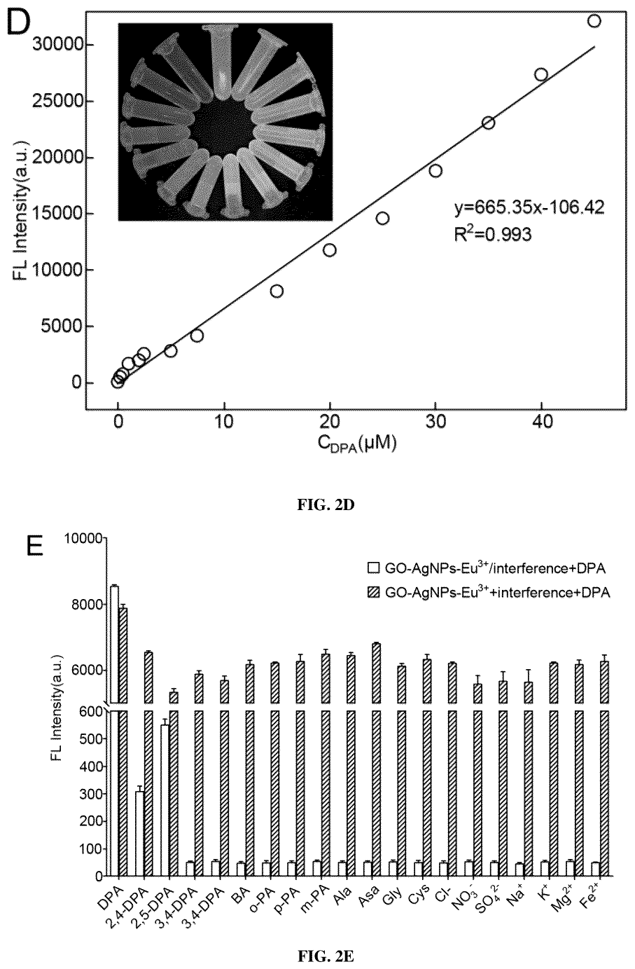

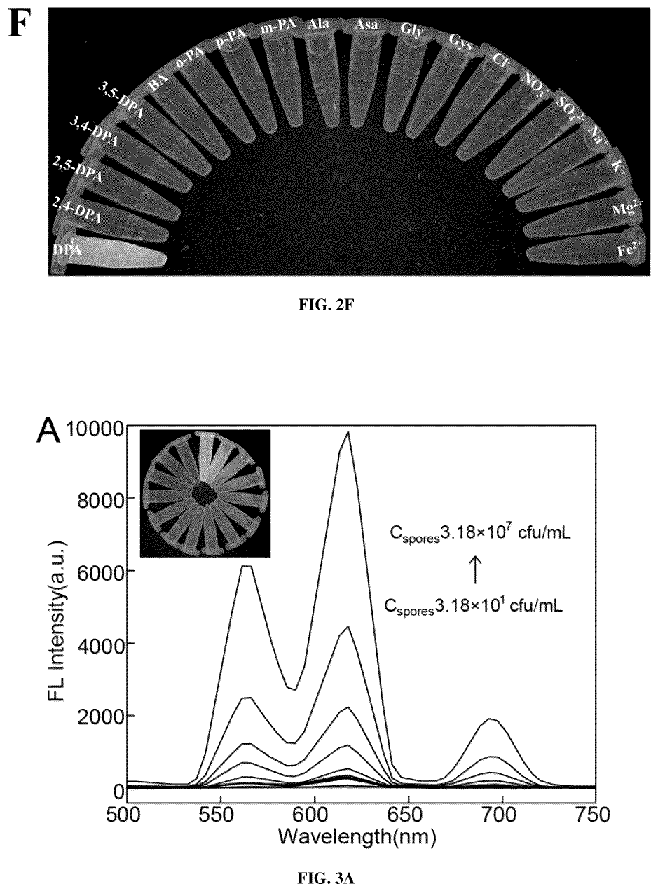

As shown in FIG. 2A, GO, GO-AgNPs, and GO-AgNPs-Eu³⁺ could not emit the characteristic fluorescence of Eu³⁺ at 616 nm, and DPA was also a non-fluorescent compound. After adding DPA into the system, the characteristic fluorescence of Eu³⁺ was emitted at 616 nm, the fluorescence of Eu³⁺ was effectively enhanced through an antenna effect (AE) and energy was transferred to lanthanide ions (Eu³⁺). By comparing the fluorescence intensity at 616 nm (FIG. 2B), it was seen that the fluorescence intensity of the GO-AgNPs-Eu³⁺ nanomaterial was much higher than that of the lanthanide ion (Eu³⁺) alone. The GO-AgNPs had the function of enhancing a fluorescence effect and hindering the coordination of water molecules. The Eu³⁺ ions could also coordinate with the groups on a surface of the GO-AgNPs, reduce the coordination with water molecules to avoid energy loss, thus enhancing the fluorescence intensity.

After adding different concentrations of DPA into the GO-AgNPs-Eu³⁺ nanomaterial, the fluorescence emission spectra were observed (FIG. 2C). When the DPA concentration was at 0 μM to 45 μM, the fluorescence emission peak at 616 nm was increased with an increase of DPA concentration, as shown in FIG. 2D. The fluorescence intensity of the GO-AgNPs-Eu³⁺ nanomaterial showed a desirable linear correlation with the DPA concentration at 0 μM to 45 μM, and a linear regression equation was y=691.09x−907.78, R²=0.99. Moreover, the LOD was calculated to be 4.62 nM according to a formula LOD=3a/k.

Different kinds of aromatic ligands, amino acids, and various common cations or anions (3,5-DPA, BA, mPA, p-PA, o-PA, 2,4-DPA, 2,5-DPA, 3,4-DPA, Asa, Ala, Cys, Gly, K⁺, Fe³⁺, Mg²⁺, Na⁺, NO₃⁻, Cl⁻, and SO₄²⁻) were examined. Under the same detection conditions (FIG. 2E), only DPA caused a signal response at 616 nm, and no interfering substance could cause a significant fluorescence change. FIG. 2F also confirmed that other interfering substances could not cause a significant color change under UV light (254 nm), and only the DPA solution emitted a significant fluorescence of Eu³⁺. The GO-AgNPs-Eu³⁺ nanomaterial had high selectivity due to the high coordination ability of DPA and Eu³⁺ in a tridentate chelation manner.

### Example 3

### Verification of the GO-AgNPs-Eu³⁺ Fluorescent Probe Prepared in Example 1 for Quantitative Detection of DPA in Spores

Preparation of C. sporogenes spores: Frozen C. sporogenes spores porcelain beads were streaked on NA medium to obtain single colonies of C. sporogenes, which were selected and transferred to 20 mL of RCM medium and cultured for 6 h, and then 200 μL of a resulting culture product was injected into RCM solid medium and cultured for 3 d to 7 d. The spores were collected by centrifugation (8,000 rpm, 10 min, and 4° C.), washed 5 to 7 times with cold sterile deionized water to remove impurities such as vegetative bodies in a resulting spore suspension, and examined again under a microscope. The spores could be used only when ≥95% of the spores in the field of view were transparent and there were no visible small impurities.

Preparation of B. subtilis spores: B. subtilis spores porcelain beads were streaked on LB plate overnight to grow single colonies. After activation for three generations, fresh single colonies were selected and transferred into 20 mL of LB liquid medium to allow overnight culture (200 rpm, 37° C.) until OD₆₀₀ was 1.2 to 1.5. Then, the colonies were transferred into spore growth-promoting medium DSM at a ratio of 1:100 to allow culture (200 rpm, 37° C.). The spores were collected by the method above.

Preparation of B. cereus spores: B. cereus spores porcelain beads were streaked on NA plate overnight to grow single colonies, the single colonies were selected and transferred to 20 mL of liquid nutrient broth medium to allow culture (200 rpm, 37° C.) for about 6 h, and then transferred to nutrient agar plate supplemented with 10.05 g/L manganese sulfate tetrahydrate at a ratio of 1:100 to allow culture for 2 d to 7 d. The spores were collected by the method above.

Preparation of B. thuringiensis spores: B. thuringiensis spores were streaked on LB plate overnight to grow single colonies, the single colonies were selected and transferred to 20 mL of LB liquid medium to allow overnight culture until OD₆₀₀ was 1.2 to 1.5, and 200 μL of a resulting culture product was injected into ICPM solid medium to allow culture for 3 d to 7 d. The spores were collected by the method above.

The concentration of the spores was initially adjusted to OD₆₀₀=0.5, approximately 10⁷ cfu/mL. A certain volume of spore suspension was continuously diluted with sterile water, a resulting spore dilution was inoculated into a culture medium at 37° C. and cultured for 24 h, and a specific concentration of the spores was obtained by plate counting. The spores diluted into different concentrations were treated at 121° C. for 30 min to ensure the complete release of DPA.

200 μL of the spore suspension and 2 μL of the GO-AgNPs-Eu³⁺ nanomaterial (with concentration of 2 mg/mL) were added into 500 μL of Tris buffer (10 mM, pH=7.0), and a fluorescence intensity was measured after allowing to stand at room temperature for 5 min.

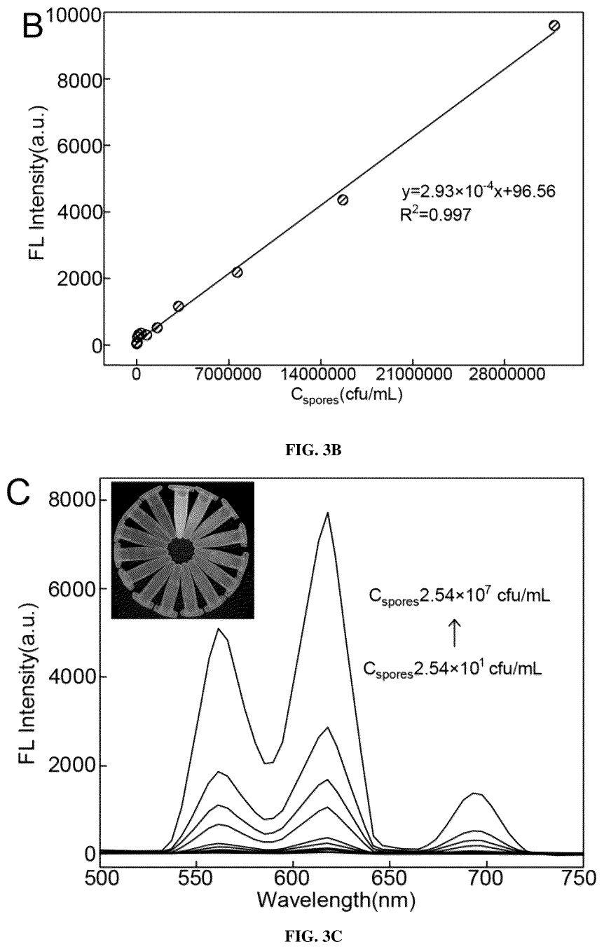

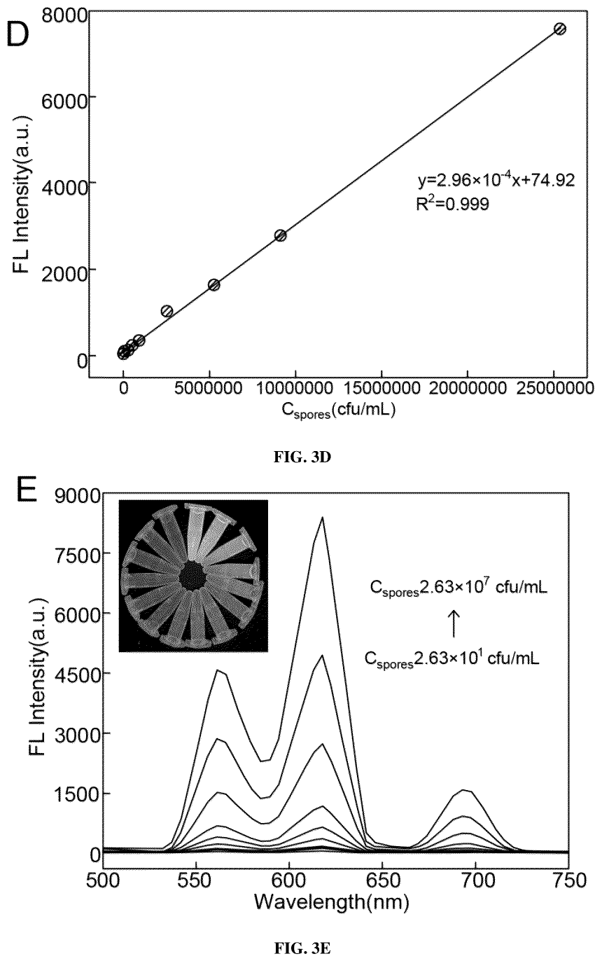

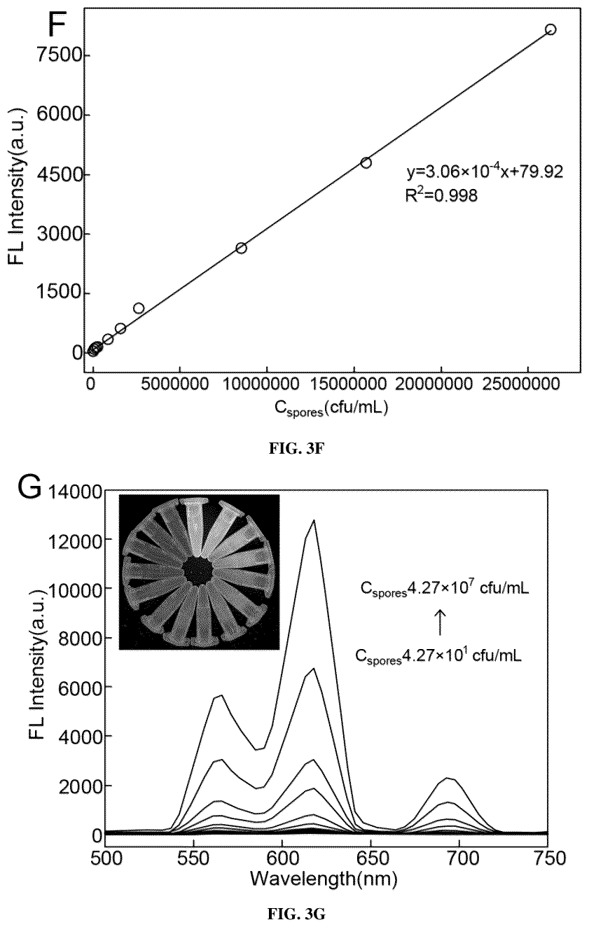

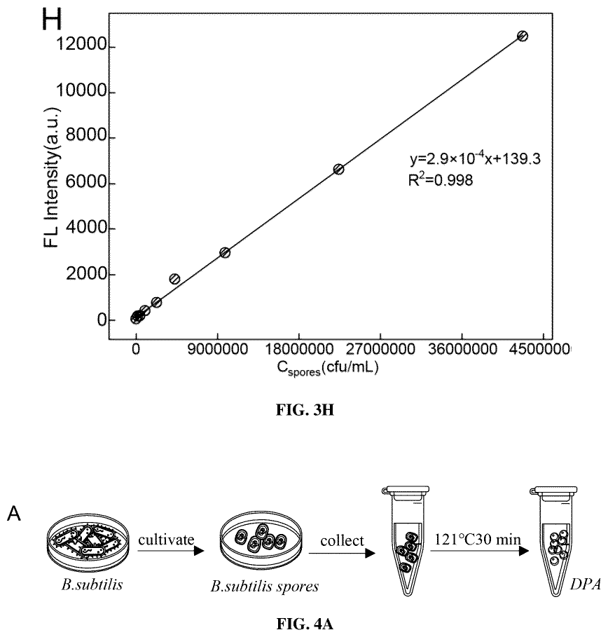

The fluorescence detection results of four foodborne spores were shown in FIGS. 3A-3H. After adding different concentrations of spore suspension, the characteristic fluorescence emission intensity of GO-AgNPs-Eu³⁺ nanomaterial was increased with the increase of spore concentration. A linear relationship between the concentration and fluorescence intensity of C. sporogenes spores was established in the concentration range of 3.18×10¹ cfu/mL to 3.18×10⁷ cfu/mL, showing that the C. sporogenes spores showed a desirable linear relationship at 10⁴ to 10⁷. The linear equation was y=2.93×10−4x+96.56, R²=0.997. According to the LOD calculation formula, the LOD of C. sporogenes spores was calculated to be 2.37×10⁴ cfu/mL. The LODs of B. subtilis spores (2.54×10¹ cfu/mL to 2.54×10⁷ cfu/mL), B. cereus spores (2.63×10¹ cfu/mL to 2.63×10⁷ cfu/mL), and B. thuringiensis spores (4.27×10¹ cfu/mL to 4.27×10⁷ cfu/mL) were also detected using the formula, and a linear relationship between the different concentrations of the three spores and the fluorescence intensity was established. The results proved that the three spores all showed a desirable linear relationship at 10⁴ to 10⁷. The linear equations of the three spores were y=2.96×10−4x+74.92 (R²=0.999), y=3.06×10−4x+79.92 (R²=0.998), and y=2.9×10−4x+139.3 (R²=0.998), and the LODs were 2.6×10⁴ cfu/mL, 2.52×10⁴ cfu/mL, and 2.66×10⁴ cfu/mL, respectively.

### Example 4

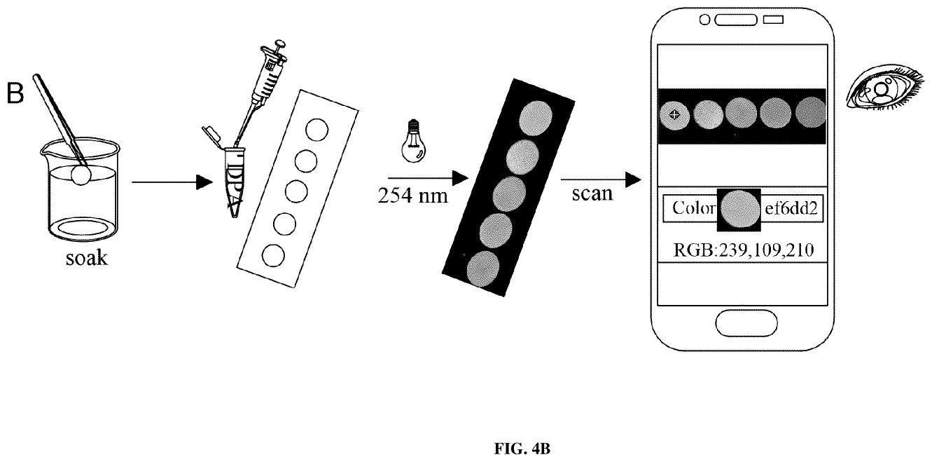

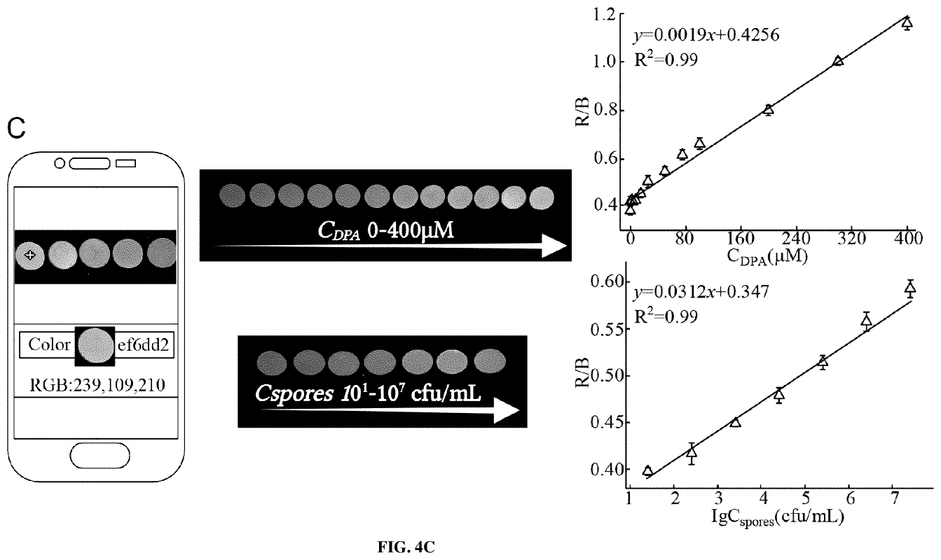

### Verification and Use of a Visual Paper-Based Sensor Based on the GO-AgNPs-Eu³⁺ Nanomaterial Shown in FIGS. 4A-4C.

The concentration of the spores was initially adjusted to OD₆₀₀=0.5, approximately 10⁷ cfu/mL. A certain volume of spore suspension was continuously diluted with sterile water, a resulting spore dilution was inoculated into a culture medium at 37° C. and cultured for 24 h, and a specific concentration of the spores was obtained by plate counting. The spores diluted into different concentrations were treated at 121° C. for 30 min to ensure the complete release of DPA. 200 μL of the spore suspension and 2 μL of the GO-AgNPs-Eu³⁺ nanomaterial (with concentration of 2 mg/mL) were added into 500 μL of Tris buffer (10 mM, pH=7.0), and a fluorescence intensity was measured after allowing to stand at room temperature for 5 min.

A PVDF microporous membrane (purchased from DELVSTLAB, a hydrophilic organic filter membrane with a pore size of 0.45 μM and a diameter of 13 mm) as a substrate was immersed in a Tris buffer containing the GO-AgNPs-Eu³⁺ fluorescent probe (where the Tris buffer had a concentration of 10 mM, pH=7.0, and the GO-AgNPs-Eu³⁺ fluorescent probe had a concentration of 5 mg/mL in the Tris buffer), incubated for 20 min, taken out, and naturally air-dried to obtain a PVDF microporous membrane-modified paper-based sensor.

20 μL of DPA solutions of different concentrations were added dropwise onto the PVDF microporous membrane, photos were taken with a smartphone, and visual analysis was conducted with naked eyes using a handheld UV lamp under irradiation of 254 nm. As shown in FIG. 4B, the color of the test paper showed dynamic changes of RGB AR, AG, and AB as concentration of DPA increased. For digital quantification, a color recognition app installed on a smartphone was used as a signal reader and analyzer. The color intensity of RGB in the fluorescent color image was digitized, and the RGB change value was calculated to allow semi-quantitative analysis.

20 μL of DPA solutions (0 μM to 400 μM) were added dropwise onto the PVDF microporous membrane-modified paper-based sensor and then dried. As shown in FIG. 5C, under 254 nm UV irradiation, the fluorescence color on the PVDF microporous membrane showed dynamic changes of RGB AR, AG, and AB as the concentration of DPA increased. Moreover, the concentration of DPA (0 μM to 400 μM) could be reflected by a ratio of R to B values (R/B), indicating that the concentration of DPA showed a desirable linear relationship with G/B (R²=0.99). The corresponding linear regression equation was y (R/B)=0.00019x (C_{DPA})+0.4256, and the LOD was 13.1 μM.

Taking B. subtilis spores as an example, different concentrations of B. subtilis spores (2.54×10¹ cfu/mL to 2.54×10⁷ cfu/mL) were treated at 121° C. for 30 min to completely release DPA, and a flow chart of DPA release from B. subtilis spores was shown in FIG. 4A. The GO-AgNPs-Eu³⁺ nanomaterial-loaded paper-based sensor was used to test DPA in spore metabolites of different concentrations. As shown in FIG. 4C, with an increase in the concentration of B. subtilis spores, the fluorescence color on the PVDF microporous membrane showed dynamic changes of RGB AR, AG, and AB under UV irradiation, indicating that the designed paper-based sensor had the ability to visually detect real spores. Moreover, a logarithmic value of spore concentration had a desirable linear relationship with G/B (R²=0.99). The corresponding linear regression equation was y (R/B)=0.0312x (lgCₛₚₒᵣₑₛ)+0.347, and the LOD was 6.3 cfu/mL.

The above shows and illustrates the basic principles, main features, and advantages of the present disclosure. It should be understood by those skilled in the art that, the present disclosure is not limited by the above examples, and the above examples and the description only illustrate the principle of the present disclosure. Various changes and modifications may be made to the present disclosure without departing from the spirit and scope of the present disclosure, and such changes and modifications all fall within the claimed scope of the present disclosure. The claimed protection scope of the present disclosure is defined by the appended claims and equivalents thereof.

What is claimed is:

1. A preparation method of a graphene oxide (GO)-silver nanoparticles (AgNPs)-Eu³⁺ fluorescent probe, comprising the following steps:
(1) preparing GO;
(2) preparing ethylenediaminetetraacetic acid dianhydride (EDTAD)-modified GO-AgNPs using the GO; and
(3) preparing the GO-AgNPs-Eu³⁺ fluorescent probe: dispersing the EDTAD-modified GO-AgNPs in ultrapure water, adding an Eu(NO₃)₃·6H₂O aqueous solution dropwise in an ultrasonic environment and stirring for a period of time; collecting a product obtained by centrifugation, washing the product with deionized water for multiple times, and then resuspending the product in the ultrapure water to obtain the GO-AgNPs-Eu³⁺ fluorescent probe solution.

2. The preparation method of a GO-AgNPs-Eu³⁺ fluorescent probe according to claim 1, wherein a preparation process of the GO in step (1) comprises: adding 0.5 g of a graphite flake and 0.25 g of sodium nitrate into 50 mL of concentrated sulfuric acid, cooling an obtained mixture to 0° C., adding potassium permanganate and stirring at a room temperature for 30 min, adding 25 mL of the ultrapure water and 2 mL of hydrogen peroxide with a mass fraction of 30% at 98° C. to allow heat preservation for 15 min, washing an obtained reaction product with 0.1 M hydrochloric acid and water in sequence, and then vacuum drying at 60° C. for 12 h to obtain the GO.

3. The preparation method of a GO-AgNPs-Eu³⁺ fluorescent probe according to claim 1, wherein a preparation process of the EDTAD-modified GO-AgNPs in step (2) comprises: adding 200 μL of a 0.5 mg/mL GO aqueous solution into 19 mL of the ultrapure water and stirring continuously for 15 min, and then adding 0.3 mL of a 10 mM silver nitrate solution and stirring continuously for 30 min; slowly adding 50 μL of a 0.01 M sodium borohydride solution and stirring continuously for 4 h; heating an obtained solution to 70° C., adding 250 mL of the ultrapure water, 25 mL of a 1,2-bis(2-aminoethoxy)ethane solution, and 500 mg of potassium hydroxide in sequence and stirring vigorously for 24 h; adding 50 mL of a 0.5 M sodium borohydride solution to allow a reaction at 70° C. for 2 h, and then collecting a precipitate obtained by centrifugation and washing the precipitate thoroughly with water; dispersing 10 mg of the precipitate ultrasonically in 5 mL of a sodium bicarbonate buffer, adding 80 mg of EDTAD and stirring for 2 h, and subjecting obtained nanoparticles (NPs) to centrifugal separation, washing, and drying to obtain the EDTAD-modified GO-AgNPs.

4. The preparation method of a GO-AgNPs-Eu³⁺ fluorescent probe according to claim 1, wherein the Eu(NO₃)₃·6H₂O aqueous solution has a concentration of 0.01 M, 5 mL of the ultrapure water and 5 mL of the Eu(NO₃)₃·6H₂O aqueous solution are required based on 10 mg of the EDTAD-modified GO-AgNPs, the stirring is conducted for 3 h, and a solution of the GO-AgNPs-Eu³⁺ fluorescent probe has a concentration of 2 mg/mL in step (3).

5. A GO-AgNPs-Eu³⁺ fluorescent probe prepared by the preparation method according to claim 1.

6. A preparation method of a paper-based sensor, comprising: immersing a polyvinylidene fluoride (PVDF) microporous membrane as a substrate into a Tris buffer of the GO-AgNPs-Eu³⁺ fluorescent probe according to claim 5, conducting incubation for a period of time, and then drying naturally in the air to obtain a PVDF microporous membrane-modified paper-based sensor.

7. The preparation method of a paper-based sensor according to claim 6, wherein the Tris buffer has a concentration of 10 mM and a pH value of 7.0, and the GO-AgNPs-Eu³⁺ fluorescent probe has a concentration of 5 mg/mL and is incubated for 20 min in the Tris buffer.

8. A method for rapid detection of a foodborne spore using the GO-AgNPs-Eu³⁺ fluorescent probe according to claim 5.

9. A method for rapid detection of a foodborne spore using a PVDF microporous membrane-modified paper-based sensor prepared by the preparation method according to claim 6.

10. The method for rapid detection of a foodborne spore according to claim 9, wherein the PVDF microporous membrane-modified paper-based sensor loaded with the GO-AgNPs-Eu³⁺ fluorescent probe is combined with a smartphone to allow on-site visual detection of spore 2,6-dipicolinic acid (DPA), and spores in milk and water are verified through a dual-function platform based on fluorescence and the paper-based sensor.

11. The GO-AgNPs-Eu³⁺ fluorescent probe according to claim 5, wherein a preparation process of the GO in step (1) comprises: adding 0.5 g of a graphite flake and 0.25 g of sodium nitrate into 50 mL of concentrated sulfuric acid, cooling an obtained mixture to 0° C., adding potassium permanganate and stirring at a room temperature for 30 min, adding 25 mL of the ultrapure water and 2 mL of hydrogen peroxide with a mass fraction of 30% at 98° C. to allow heat preservation for 15 min, washing an obtained reaction product with 0.1 M hydrochloric acid and water in sequence, and then vacuum drying at 60° C. for 12 h to obtain the GO.

12. The GO-AgNPs-Eu³⁺ fluorescent probe according to claim 5, wherein a preparation process of the EDTAD-modified GO-AgNPs in step (2) comprises: adding 200 μL of a 0.5 mg/mL GO aqueous solution into 19 mL of the ultrapure water and stirring continuously for 15 min, and then adding 0.3 mL of a 10 mM silver nitrate solution and stirring continuously for 30 min; slowly adding 50 μL of a 0.01 M sodium borohydride solution and stirring continuously for 4 h; heating an obtained solution to 70° C., adding 250 mL of the ultrapure water, 25 mL of a 1,2-bis(2-aminoethoxy)ethane solution, and 500 mg of potassium hydroxide in sequence and stirring vigorously for 24 h; adding 50 mL of a 0.5 M sodium borohydride solution to allow a reaction at 70° C. for 2 h, and then collecting a precipitate obtained by centrifugation and washing the precipitate thoroughly with water; dispersing 10 mg of the precipitate ultrasonically in 5 mL of a sodium bicarbonate buffer, adding 80 mg of EDTAD and stirring for 2 h, and subjecting obtained nanoparticles (NPs) to centrifugal separation, washing, and drying to obtain the EDTAD-modified GO-AgNPs.

13. The GO-AgNPs-Eu³⁺ fluorescent probe according to claim 5, wherein the Eu(NO₃)₃·6H₂O aqueous solution has a concentration of 0.01 M, 5 mL of the ultrapure water and 5 mL of the Eu(NO₃)₃·6H₂O aqueous solution are required based on 10 mg of the EDTAD-modified GO-AgNPs, the stirring is conducted for 3 h, and a solution of the GO-AgNPs-Eu³⁺ fluorescent probe has a concentration of 2 mg/mL in step (3).

14. A method for rapid detection of a foodborne spore using a PVDF microporous membrane-modified paper-based sensor prepared by the preparation method according to claim 7.
